(19) United States
(12) Patent Application Publication
(10) Pub. No.: US 2005/0260742 A1
(43) Pub. Date: Nov. 24, 2005

(54) Culture-vessel adaptor and culture treatment apparatus

(75) Inventor: Sadahiro Watanabe, Tokyo (JP)

Correspondence Address: Thomas Spinelli;Scully, Scott, Murphy & Presser, 400 Garden City Plaza, Garden City, NY, 11530, US

(73) Assignee: OLYMPUS CORPORATION, TOKYO (JP)

(21) Appl. No.: 11/130,782

(22) Filed: May 17, 2005

(30) Foreign Application Priority Data
May 24, 2004 (JP) 2004-153704

(51) Int. Cl. C12M 1/00

(52) U.S. Cl. 435/287.3; 435/283.1; 435/288.3

(57) ABSTRACT

A culture-vessel adaptor that is detachably fixed to the exterior of a culture vessel capable of holding a culture medium containing a specimen is provided. The culture-vessel adaptor includes a vessel mounting part where the culture vessel is mounted; and a protruding part disposed so as to extend outward from a side surface of the culture vessel when the culture vessel in mounted in the vessel mounting part. Handling of culture vessels becomes easy by an automated mechanism such as a robot, with a straightforward and low-cost method, and the culture vessels can be reliably prevented from being dropped.

### BACKGROUND OF THE INVENTION

1. Field of the Invention

The present invention relates to a culture-vessel adaptor and to a culture treatment apparatus.

2. Description of Related Art

Conventionally known culture vessels include flask-type culture vessels (see for example Japanese Unexamined Patent Application Publication No. HEI-10-179137, FIGS. 1 and 8 therein) and circular Petri dish culture vessels (see for example, Japanese Unexamined Patent Application Publication No. HEI-10-210966, FIG. 1 therein).

These culture vessels are used mainly in carrying out manual culture treatment, for example, replacing the culture medium or subculture treatment. They are not used in automatic culture treatment.

The reason is because the circular culture vessels have a structure wherein the side surfaces extend upwards from the bottom surface substantially without changing their shape. Therefore, if these culture vessels are to be manipulated by an automated mechanism, a robot for handling the culture vessels must be provided with a hand for gripping the vessels from both sides.

However, this type of hand is a complex device. Also, since the hand is designed to prevent the culture vessel from being dropped by using frictional force, there is a problem in that it is difficult to reliably handle the vessels. That is to say, if the frictional force is reduced for some reason, the culture vessel may be dropped from the hand, which results in the problem that the carefully incubated specimen, such as cells, is wasted or some of the specimen splashing out may contaminate other specimens.

In the case where the culture vessel contains culture medium and in the case where culture vessel is empty, the weight thereof is quite different, and so the frictional force required to grip the vessel without dropping it varies, making control difficult. In addition, when a crack or the like occurs in the culture vessel, the specimen contained inside may leak, resulting in the problem of the specimen splashing out.

On the other hand, if a special culture vessel having a projection in the side surface is employed, the problems described above can be avoided. However, such a specially designed culture vessel lacks versatility, thus resulting in the problem of increased costs.

### BRIEF SUMMARY OF THE INVENTION

In light of the circumstances described above, it is an object of the present invention to provide a culture-vessel adaptor and a culture treatment apparatus that allow easy handling of culture vessels by an automated mechanism such as a robot, with a straightforward and low-cost method, and that can reliably prevent the culture vessels from being dropped.

To realize the above-described object, the present invention provides the solutions described below.

The present invention provides a culture-vessel adaptor that is detachably fixed to the exterior of a culture vessel capable of holding a culture medium containing a specimen, the culture-vessel adaptor including a vessel mounting part where the culture vessel is mounted; and a protruding part disposed so as to extend outward from a side surface of the culture vessel when the culture vessel is mounted in the vessel mounting part.

According to this aspect of the invention, when the culture vessel is mounted in the vessel mounting part, since the protruding part is disposed so as to extend outward from the side surface of the vessel, the culture-vessel adaptor can be lifted while supported from below this protruding part, which allows the culture vessel to be manipulated. In other words, the culture vessel is not lifted by means of a frictional force by gripping it from the left and right sides, but is supported from below. Therefore, the culture vessel can be easily and stably handled even with an automatic mechanism such as a robot. In addition, a specially designed culture vessel is not required, and regular commercially available culture vessels can thus be used.

The aspect of the invention described above preferably also includes a peripheral wall that is connected to the vessel mounting part around the entire circumference of the vessel mounting part and that is disposed at a certain distance outside the side surface of the culture vessel when the culture vessel is mounted in the vessel mounting part.

With this configuration, even if the culture medium including the specimen flows out due to breakage of the culture vessel or shaking, the spillage is caught by the vessel-mounting part, and in addition, can be prevented from flowing outside by the peripheral wall.

In the aspect of the invention described above, the protruding part may be formed in the shape of a flange.

With this configuration, the position at which the protruding part is supported from below can be freely selected within the width of the protruding part. Therefore, when manipulating the culture vessel using an automated mechanism such as a robot, the positioning need be performed only roughly.

In the aspect of the invention described above, a fitting portion for fitting the culture vessel is preferably provided in the vessel mounting part.

By fitting the culture vessel into the fitting part when mounting the culture vessel in the vessel-mounting part, movement of the culture vessel in the vessel-mounting part is prevented, even if the vessel-mounting part is tilted. Therefore, when tilting the culture vessel to draw up the culture medium at the bottom surface with a pipette, it is possible to prevent the drawing up position from changing, which allows the procedure to be easily automated. In addition, when the culture vessel is agitated to mix the liquid inside, since the culture vessel and the culture-vessel adaptor are relatively fixed, the mixing can be carried out more effectively.

In the aspect of the invention described above, the culture-vessel adaptor may be formed of a heat-resistant plastic material.

With this configuration, sterilization can be performed by heating in an autoclave, for example, which allows the vessels to be re-used.

In the aspect of the invention described above, at least the vessel mounting part is preferably formed of a transparent material, or a through-hole is preferably formed in the vessel mounting part.

By forming the vessel-mounting part of a transparent material or by forming a through-hole in the vessel-mounting part, an inverted microscope can be used to examine the specimen inside the culture vessel from below, via the transparent vessel-mounting part or via the through-hole.

Furthermore, the present invention provides a culture treatment apparatus including a treatment chamber containing a treatment apparatus that opens a lid of a culture vessel containing a specimen and that performs predetermined treatment on the specimen in the culture vessel; and a handling apparatus, provided in the treatment chamber, for transferring the culture-vessel adaptor, in which the culture vessel is mounted, by engaging with the protruding part from below.

According to this aspect of the invention, since the culture vessels are transferred by a handling apparatus that engages with the protruding part of the culture-vessel adaptor from below, rather than by gripping the side walls of the culture vessels, the culture vessels can be transferred quickly and stably.

According to the present invention, culture vessels can be manipulated while being supported from below, which allows them to be easily handled with an automated mechanism such as a robot. In addition, since commercially available cost-efficient culture vessels can be employed, rather than specially designed culture vessels, an advantage is afforded in that the costs can be reduced.

### BRIEF DESCRIPTION OF THE SEVERAL VIEWS OF THE DRAWINGS

FIG. 1 is a longitudinal sectional view of a culture-vessel adaptor according to an embodiment of the present invention.

FIG. 2 is a longitudinal sectional view showing the culture-vessel adaptor of FIG. 1 when a culture vessel is mounted therein.

FIG. 3 is a longitudinal sectional view showing the culture-vessel adaptor of FIG. 2, in which a culture vessel is mounted, while being titled.

FIG. 4 is a longitudinal sectional view showing a first modification of the culture-vessel adaptor in FIG. 1.

FIG. 5 is a longitudinal sectional view showing a second modification of the culture-vessel adaptor in FIG. 1.

FIG. 6 is a longitudinal sectional view showing a third modification of the culture-vessel adaptor in FIG. 1.

FIG. 7 is a longitudinal sectional view showing a fourth modification of the culture-vessel adaptor in FIG. 1 FIG. 8 is a perspective view showing a culture treatment apparatus according to an embodiment of the present invention and an automatic culture apparatus employing the same.

FIG. 9 is a longitudinal sectional view schematically showing a first space in the automatic culture apparatus in FIG. 8.

FIG. 10 is a plan view schematically showing the first space in the automatic culture apparatus in FIG. 8.

FIG. 11 is a perspective view of devices above a second compartment wall, while omitting a first compartment wall, of the culture treatment apparatus of the automatic culture apparatus in FIG. 8.

FIG. 12 is a longitudinal sectional view of an example of an attachment structure of a duct that connects to a disposal container of the culture treatment apparatus in the automatic culture apparatus in FIG. 8.

FIG. 13 is a perspective view of an apparatus disposed inside a bottommost space, omitting the first and second compartment walls of the culture treatment apparatus in the automatic culture apparatus in FIG. 8.

### DETAILED DESCRIPTION OF THE INVENTION

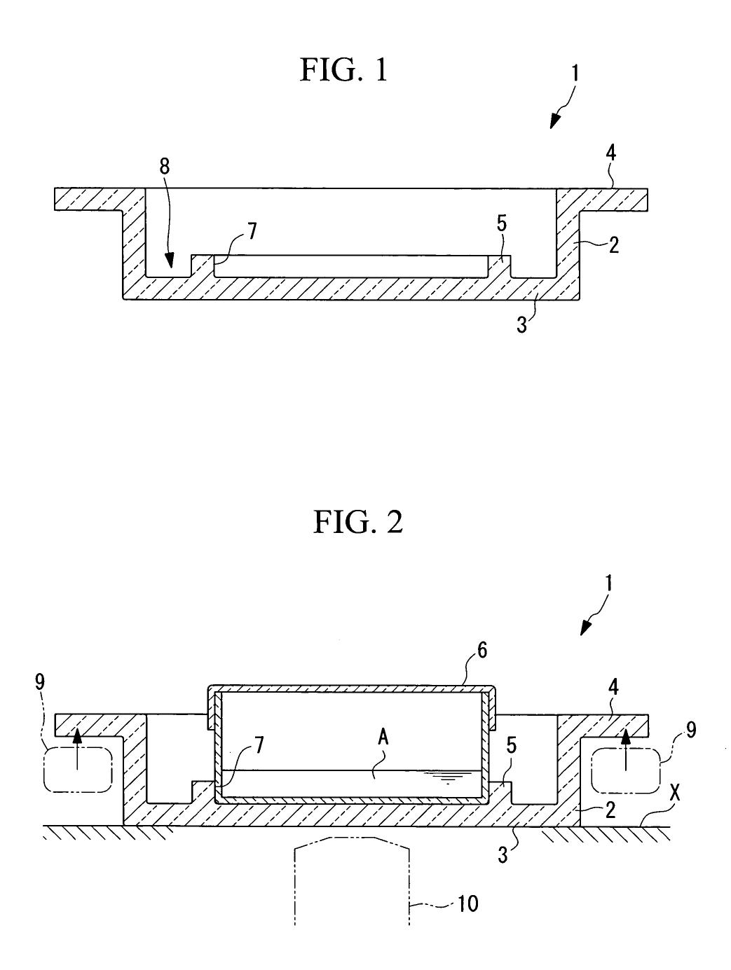

A culture-vessel adaptor 1 according to an embodiment of the present invention is described below with reference to FIGS. 1 to 3.

As shown in FIG. 1, the culture-vessel adaptor 1 according to this embodiment includes a peripheral wall 2 formed substantially in the shape of a circle; a bottom surface 3 that closes off one end of the peripheral wall 2; and a flat ring-shaped protruding part 4 that extends radially outwards from the other end of the peripheral wall 2 to form a flange.

A circular raised portion 5 that protrudes upwards is provided on the top face of the bottom surface 3, and a fitting portion 7 for fitting a culture vessel 6, which is formed of a circular Petri dish, as shown in FIG. 2, is provided on the inner side of the raised portion 5. The culture vessel 6 is mounted such that the lower surface thereof is placed in contact with the bottom surface 3. Therefore, a vessel mounting part for mounting the culture vessel 6 (hereinafter referred to as a vessel mounting part 3) is formed by the bottom surface 3.

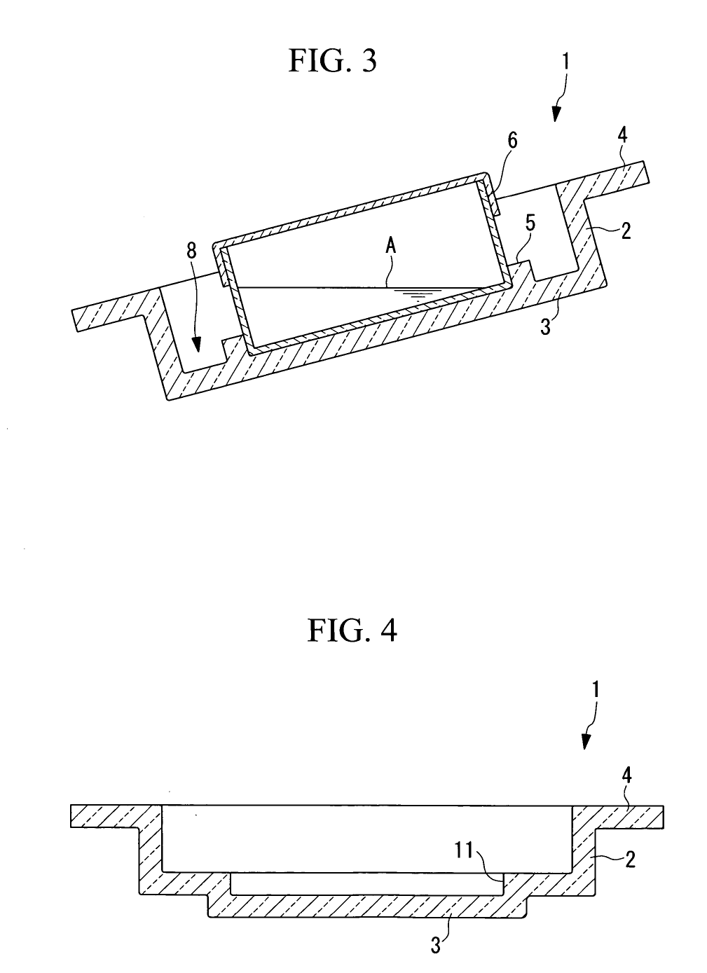

By engaging with the outer surface of the culture vessel 6, the raised portion 5 can hold the culture vessel 6 so that it does not move in a direction parallel to the upper surface of the vessel-mounting part 3 with respect to the culture-vessel adaptor 1. By doing so, as shown in FIG. 3, the culture vessel 6, when disposed inside the culture-vessel adaptor 1, does not move relative to the culture-vessel adaptor 1, even if the culture-vessel adaptor 1 is tilted.

The peripheral wall 2 is disposed surrounding the circumference of the vessel-mounting part 3 so as to form a ring-shaped groove 8 outside the raised portion 5. Thus, even if the specimen A, such as a culture medium, held in the culture vessel 6 leaks outside due to breakage, shaking or the like of the culture vessel 6, this structure provides a dam for causing any of the specimen A flowing out to be held in the groove 8 so that it does not splash out.

Since the protruding part 4 is provided at a position away from the bottom surface 3 in the height direction, it is disposed so as to extend radially outwards at an intermediate point in the height direction of the outer surface of the culture vessel 6 when the culture vessel 6 is mounted in the vessel mounting part 3. That is, even if the culture-vessel adaptor 1 in which the culture vessel 6 is mounted is placed such that the bottom surface 3 thereof is disposed in close contact with an installation surface X inside a storage area on a flat tray or in an incubator, the protruding part 4 is separated by a certain gap in the height direction from the installation surface X.

Therefore, as shown in FIG. 2, a robot hand 9 can be inserted into this gap, and the protruding part 4 of the culture-vessel adaptor 1 can be supported from below and lifted by the robot hand 9.

The culture-vessel adaptor 1 is formed of a transparent, heat-resistant plastic material. By using a heat-resistant material, it is possible to carry out heat treatment, such as autoclave sterilization and so forth, which enables re-use of the culture vessels after sterilization. Also, by using a transparent material, as shown by the chain line in FIG. 2, an objective lens of the inverted microscope 10 can be brought near from below, which allows the specimen, such as cells, inside the culture vessel 6 to be examined in the cultured state through the culture-vessel adaptor 1.

The operation of the culture-vessel adaptor 1 of this embodiment, with such a configuration, will be described below.

When using the culture-vessel adaptor 1 according to this embodiment, as shown in FIG. 2, the culture vessel 6 is fitted inside the ring-shaped raised portion 5 provided on the bottom surface 3, and the lower surface thereof is brought into contact with the vessel mounting part 3. The culture vessel 6 may be a commercially available circular Petri dish, but even a vessel with a different shape, such as, for example, a flask, can still be used by so long as it is formed so that it fits inside the raised portion 5.

With this configuration, as shown in FIG. 2, the robot hand 9 can be inserted below the flange-shaped protruding part 4 to lift it, which allows for easy handling of the culture vessel 6. In other words, conventionally, when handling a culture vessel 6 formed of a circular Petri dish with the robot hand 9, when gripping the culture vessel 6 from the left and right sides while placed on a flat installation surface X, the culture vessel 6 can only be lifted by the frictional force due to the gripping force. Under such circumstances, since the frictional force at the connection part between the robot hand 9 and the side surfaces of the culture vessel 6 varies depending on the surface condition or the gripping force of the robot hand, there is a problem in that the culture vessel 6 may be dropped if the gripping force is too low.

In contrast, with the culture-vessel adaptor 1 according to this embodiment, the culture vessel 6 can be lifted by applying a force from below, via the protruding part 4, the peripheral wall 2, and the vessel mounting part 3. Therefore, an advantage is afforded in that it is possible to provide stable gripping so that the culture vessel 6 is not dropped, even when the robot hand 9 and the culture-vessel adapter 1 are in contact and during uncontrollable conditions due to unexpected stopping of the robot.

Also, since the protruding part 4 supported by the robot hand 9 is formed in a ring shape, the robot hand 9 can access the culture-vessel adaptor 1 from any angle to manipulate it. Therefore, it is easy to issue operating instructions during handling, and an advantage is provided in that it is not necessary to carry out precise positioning.

In addition, with the culture-vessel adaptor 1 according to the present embodiment, the culture vessel 6 is fixed to the culture-vessel adaptor 1 so as not to move, by means of the raised portion 5 provided on the bottom surface 3. Therefore, even when tilting the culture vessel 6 to gather the culture medium at the corner for drawing off some of the culture medium with a pipette (not shown), such as when changing the culture medium, the culture vessel 6 can be stably held in the tilted position. As a result, since the position at which the pipette is located does not change, it is possible to easily carry out positioning of the pipette by automated mechanism, such as a robot or the like.

Also, the culture vessel 6 can be fixed to the culture-vessel adaptor 1 to perform stable shaking even when the culture vessel 6 is shaken to agitate a liquid held therein, such as the culture medium, trypsin, and so on.

In the culture-vessel adaptor 1 according to this embodiment, the peripheral wall 2 is formed surrounding the vessel mounting part 3, and a ring-shaped groove 8 is formed between the peripheral wall 2 and the raised portion 5. Therefore, even if the culture vessel 6 becomes broken or if the liquid A held inside, such as trypsin or a culture medium containing a specimen, leaks out of the culture vessel 6 during the shaking operation mentioned above, any leaked liquid A is completely collected in the groove 8, and can thus be prevented from splashing outside.

Furthermore, since the culture-vessel adaptor 1 according to this embodiment is formed of a transparent material, it is possible to examine the appearance of specimen inside the culture vessel 6 with the inverted microscope 10 while the culture vessel 6 is mounted in the vessel mounting part 3. Therefore, it is not necessary to remove the culture vessel 6 from the culture-vessel adaptor 1 at an examination stage, which allows the procedure to be simplified.

Also, since the material has heat resistance and can thus be sterilized in an autoclave, the culture vessel 6 can be re-used, which is cost efficient.

In the culture-vessel adaptor 1 according to the present embodiment, the groove 8 for collecting spilled liquid is formed between the peripheral wall 2 and the raised portion 5 of the vessel-mounting part 3; however, as shown in FIG. 4, a depression 11 may be employed instead of the raised portion 5 so as to provide a configuration in which the liquid A leaking out of the culture vessel 6 is completely trapped inside the peripheral wall.

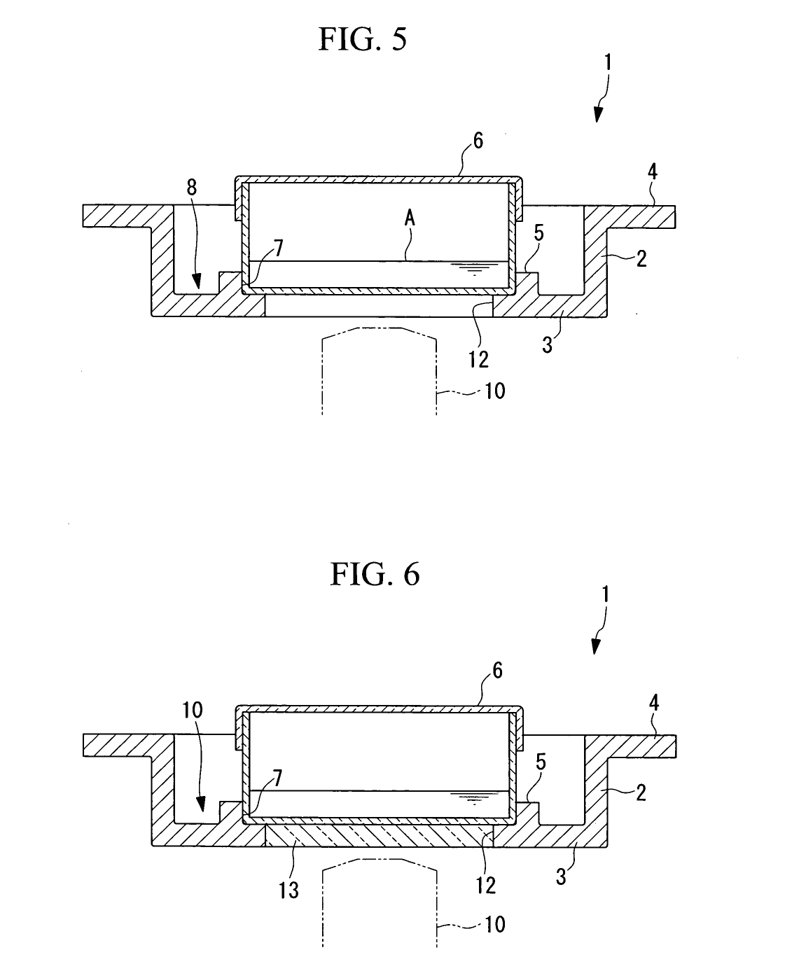

Although the entire culture-vessel adaptor 1 is formed of a transparent material, instead of this, as shown in FIG. 5, a through-hole 12 may be provided in the vessel-mounting part 3. With this arrangement, the appearance of the specimen inside the culture vessel 6 can be examined via the through-hole 12 with the inverted microscope 10. In addition, the culture-vessel adaptor 1 can be made of any material, and is not only limited to a transparent material, which allows better heat resistance, weight saving, reduced costs, and so on.

As shown in FIG. 6, it is also possible to dispose a transparent plate 13 only in the through-hole 12, to tightly seal the through-hole 12.

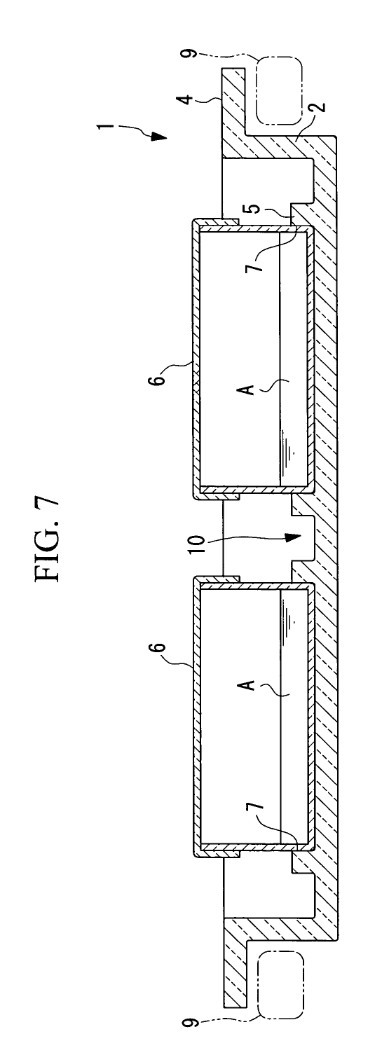

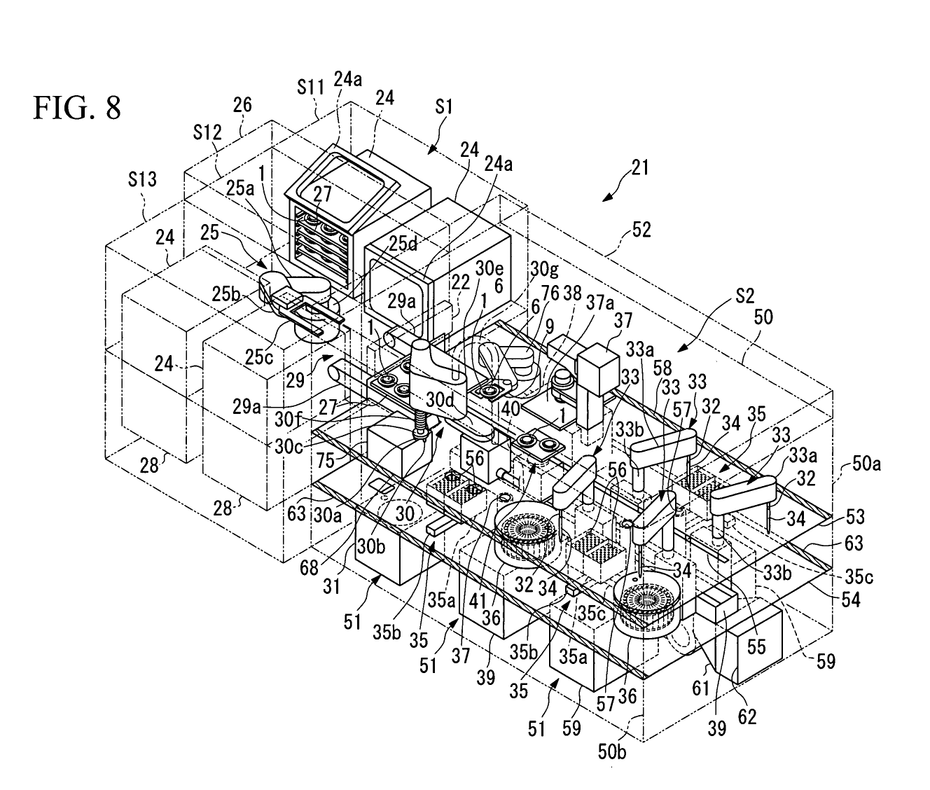

In the above embodiment, a case in which a single culture vessel is mounted in each culture-vessel adaptor 1 has been described; instead of this, however, as shown in FIG. 7, a structure in which a plurality of culture vessels are mounted may be employed. With this structure, it is possible to handle a plurality of culture vessels simultaneously. For example, during a transfer operation between individual treatment devices and during transfer to a microscope stage, it is possible to process a plurality of culture vessels simultaneously.

Therefore, the throughput can be improved by increasing the operating efficiency. Also, by mounting a plurality of culture vessels in one culture-vessel adaptor, when performing a dispensing operation, the dispensing operation can be carried out sequentially on adjacent culture vessels. Compared to the case where a chemical to be supplied to each culture vessel is drawn up and supplied to each culture vessel individually, an advantage is afforded in that the time required can be significantly shortened.

Next, a culture treatment apparatus 50 and an automatic culture apparatus 21 according to an embodiment of the present invention are described below with reference to FIGS. 8 to 13.

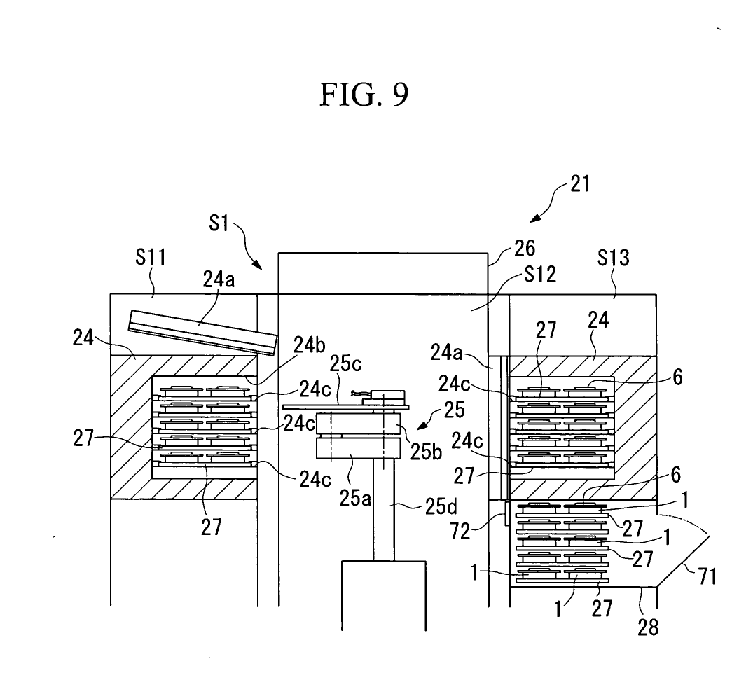

The culture treatment apparatus 50 according to this embodiment employs the automatic culture apparatus 21 shown in FIGS. 8 and 9.

As shown in FIG. 8, this automatic culture apparatus 21 includes a first space S1 and a second space S2 (treatment chamber), which communicate with each other via a shutter 22.

Two incubation chambers 24, which hold the culture vessels 6, are disposed in each of side spaces S11 and S13 inside the first space S1 to make a total of four incubation chambers 24. A transfer robot (transfer mechanism) 25 for moving the culture vessels 6 is provided in a central space S12. At the upper part of the central space S12, an air cleaning unit 26 that sends a downward flow of clean air in order to clean the air inside the central space 12 is provided.

By providing each of the four incubation chambers 24 with a door 24a facing the central space S12, two doors 24a arranged side-by-side face another two doors 24a, with a space disposed therebetween.

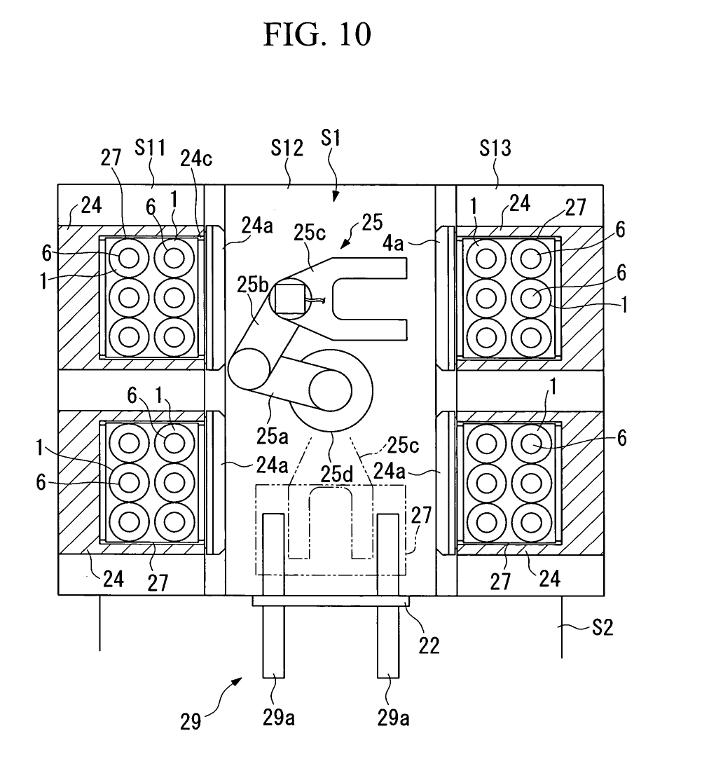

As shown in FIGS. 9 and 10, each incubation chamber 24 has an opening 24b at one side thereof, and each opening 24b is provided with the door 24a, which can open and close at the opening 24b. In the side walls facing each other at the left and right sides of the opening 24b, a plurality of rail-shaped tray holding members 24c are provided at corresponding heights. By placing trays 27 across the pairs of tray holding members 24c at the left and right sides, it is possible to store trays 27 at a plurality of levels in the height direction. The tray holding members 24c are not limited to rail-shaped members; they may be formed in any shape so long as they allow the trays 27 to be inserted and removed while being supported. Predetermined incubation conditions are maintained inside each incubation chamber 24, for example a temperature of 37±0.5° C., a humidity of 100%, and a CO₂ concentration of 5%.

On each tray 27, a plurality of, for example six, culture vessels 6 can be mounted side-by-side.

As shown in FIGS. 8 and 9, a stacker 28 that contains a plurality of unused culture vessels 6 mounted on trays 27 is disposed below each incubation chamber 24. Each stacker 28 has a door 71 that can be opened and closed at a side facing towards the outside of the first space S1 in the opposite direction to the door 24a of the incubation chamber 24. The door 71 is formed with a size large enough to open one entire side of the stacker 28.

The transfer robot 25 is positioned substantially at the center of the gap between the four incubation chambers 24. The transfer robot 25 includes a horizontally rotatable first arm 25a; a second arm 25b that is continuous with the end of the first arm 25a so as to be rotatable about a vertical axis; a hand 25c that is attached to the end of the second arm 25b so as to be rotatable about a vertical axis, and that does not have a mechanism that might degrade the environment inside the incubation chamber, such as a drive unit or power train; and a raising and lowering mechanism 25d that can raise and lower the first arm 25a, the second arm 25b, and the hand 25c. With this configuration, the transfer robot 25 can access all trays 27 inside the four incubation chambers 24, and in addition, it has a horizontal operating range such that it can pass trays 27 onto a conveyer 29 disposed between the first space S1 and the second space S2 via the shutter 22.

The conveyer 29 includes two endless belts 29a disposed at the left and right with a space therebetween larger than the width of the hand 25c of the transfer robot 25, so that the trays 27 can be placed across the endless belts 29a. In addition to the transfer robot 25 accessing all trays 27 inside the incubation chambers 24, it also has a vertical-direction operating range such that it can access a feeding port 72 provided at a position according to at least the uppermost tray in the stacker 28.

The belts 29a are not limited to endless belts.

The hand 25c is formed in a flat shape extending horizontally so that the trays 27 can be mounted thereon, and is formed with a thickness that can be inserted into the space between trays 27 when stored in the incubation chambers 24. Thus, by raising the hand 25c from the position at which it is inserted into the space between the trays 27, the trays 27 can be pushed up from below by two prongs and removed from the tray holding members 24c, and in addition, the trays 27 can be held stably.

As shown in FIG. 8, a culture treatment apparatus 50 is provided in the second space S2.

As shown in FIG. 8, the culture treatment apparatus 50 includes a specimen feeder 75, a handling robot 76, a supply robot 30, a centrifugal separator 31, dispensing robots 33, chip supply apparatuses 35, chip recovery units 51, reagent supply apparatuses 36, a microscope 37, a reservoir tank 38, a horizontal transfer mechanism 39, and a mounting stage 41.

The specimen feeder 75 feeds, for example, bone marrow taken from a patient at a medical facility to collect specimens including many mesenchymal stem cells from the supplied bone marrow. PBS (phosphate buffered saline) etc. is supplied to the provided bone marrow and agitated so as to separate a specimen including many mesenchymal stem cells from the agitated bone marrow.

The handling robot 76, which is, for example, a horizontal articulated robot like the transfer robot 25, can lift culture-vessel adaptors 1 on the tray 27 and transfer them by moving a robot hand 9 provided at the end thereof. As described above, the robot hand 9 can engage, from below, with a protruding part 4 of the culture-vessel adaptor 1 in which a culture vessel 6 is mounted so as to lift the culture-vessel adaptor 1 together with the culture vessel 6.

The supply robot 30 supplies specimens collected in the above-described specimen feeder 75 to the culture vessels 6 on the tray 27 transferred from the first space S1 by the conveyer 29 when the shutter 22 is opened, and also supplies and recovers culture medium. The centrifugal separator 31 is designed to separate cells from the culture medium in the culture dishes 6.

The dispensing robots 33 each includes an automatic pipette 32 for dispensing a variety of liquids, such as blood serum, reagents, and the like, and is capable of horizontal rotation and upward and downward motion. Four dispensing robots 33 are provided in the second space S2.

The chip supply robots 35 contain a plurality of disposable chips 34 that are attached to the automatic pipettes 32 of the dispensing robots 33 and the supply robot 30, and supply them within the operating range of the supply robot 30 and the dispensing robots 33. The chip recovery units 51 recover used chips 34 for disposal. The supplying apparatuses 36, for supplying reagents or the like, described above, hold various liquids such as blood serum and reagents in a plurality of containers. The microscope 37 allows examination of the appearance of cells inside the culture vessels 6. A plurality of the reservoir tanks 38 are provided for holding respective waste solutions discarded after replacing the reagents and culture media. The horizontal transfer mechanism 39 moves the culture vessels 6 so that the culture vessels 6 can be handed over between the conveyer 29 and the robots 30 and 33. The mounting stage 41 is attached to a slider 40 of the horizontal transfer mechanism 39 and is designed such that the received culture vessels 6 are mounted thereon.

An air cleaning unit 52 (ventilation unit) 52 producing a downward flow of clean air for cleaning the air inside the second space S2 is also provided inside the second space S2.

The culture treatment apparatus 50 constructed in the second space S2 is partitioned into three spaces S21, S221, and S222 arranged in the vertical direction by means of a first compartment wall 53 and a second compartment wall 54. The first compartment wall 53, which is provided at a central position in the height direction, divides the second space S2 into an upper space S21 and a lower space S22, and the second compartment wall 54 divides the lower space S22 formed by the first compartment wall 53 into spaces S221 and S222.

The first compartment wall 53 is positioned at the height of the conveyer 29, and the mounting stage 41, the supply robot 30, arms 33a of the dispensing robots 33, mechanisms at or above an XY table 37a of the microscope 37, etc. are disposed in the upper space S21 above the first compartment wall 53. This is because these devices are devices necessary for moving the culture vessels 6 and devices necessary for accessing the culture vessels 6 from the openings at the top thereof. The upper surfaces of the supply apparatuses 36 for supplying reagents etc. are also exposed at the upper surface of the first compartment wall 53, but this is to form feeding ports 36c for feeding the chips 34 into the upper space S21.

An elongated hole 55 for connecting the mounting stage 41 with the horizontal transfer mechanism 39 in the lower space S22 is formed in the first compartment wall 53 for moving the mounting stage 41 in the upper space S21. Also formed in the first compartment wall 53 are through-holes 56 for extracting chips 34 from the chip supply apparatuses 35 disposed in the space S221 below the first compartment wall 53, and disposal holes 57 for disposing of used chips 34. Furthermore, ventilation holes 58 passing through from top to bottom are provided in the first compartment wall 53 parallel to the side walls 50a and 50b thereof.

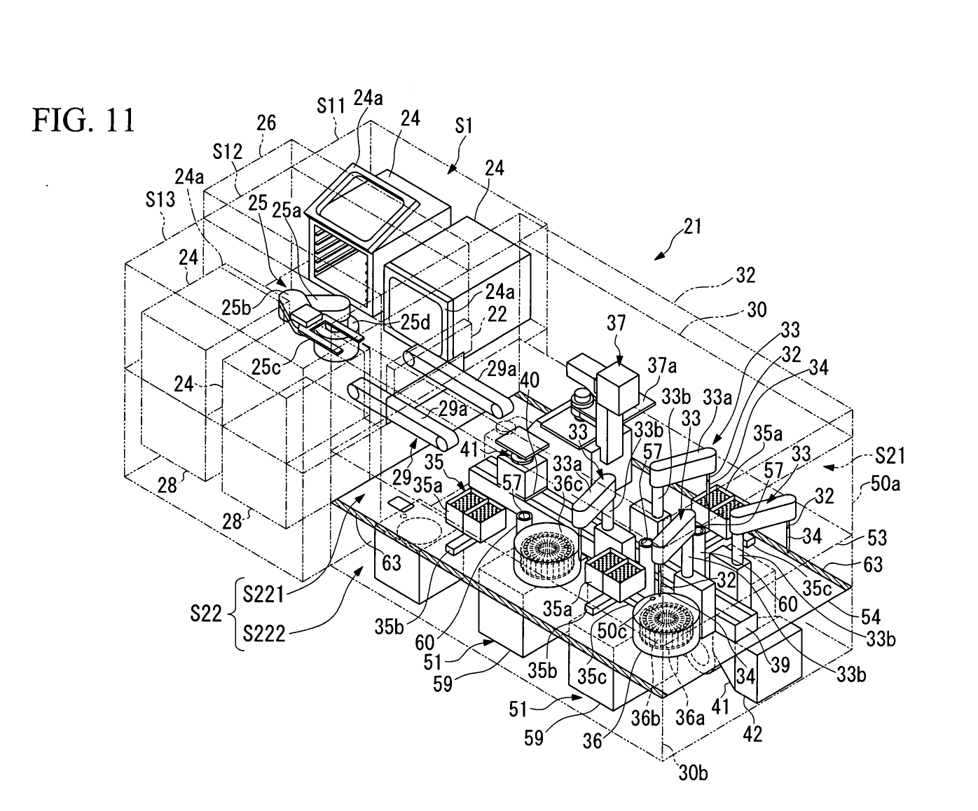

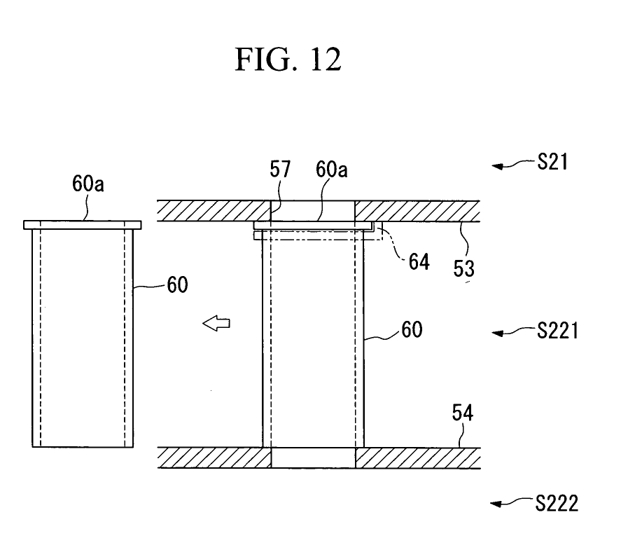

As shown in FIG. 11, the main bodies of the dispensing robots 33, the chip supply apparatuses 35, the reagent supplying apparatuses 36, the lower part of the XY table 37a of the microscope 37, the horizontal transfer mechanism 39, and ducts 60 connecting the disposal holes 57 and disposal containers 59 of the chip recovery units 51 are provided in the space S221 between the first compartment wall 53 and the second compartment wall 54. As shown in FIG. 12, each duct 60 is formed, for example, of flange 60a at the upper end, and by catching it on a hook 64 provided underneath the first compartment wall 53, it may be removably provided between the first compartment wall 53 and the second compartment wall 54. Ventilation holes 63 (shaded areas) passing through from top to bottom parallel to the side walls 50a and 50b are provided adjacent to the side walls 50a and 50b of the second compartment wall 54.

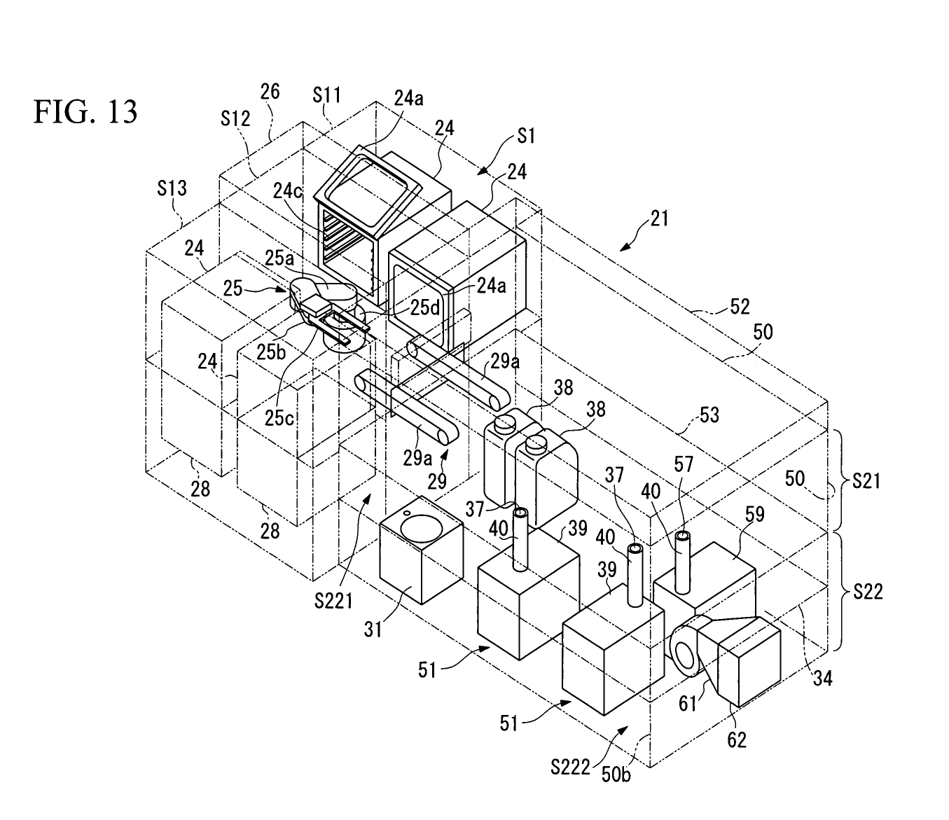

As shown in FIG. 13, the centrifugal separator 31, the reservoir tanks 38, the disposal containers 59, and an exhaust fan 61 are disposed in the space S222 below the second compartment wall 54. A filter 62, such as a HEPA filter, is provided at the outlet of the exhaust fan 61 to filter the exhausted air.

As shown in FIG. 8 for example, the supply robot 30, which is a horizontally articulated robot, includes a head 30c having two types of automatic pipettes 30a and 30b; two horizontally revolving arms 30d and 30e; and a raising and lowering mechanism 30f that raises and lowers the head 30c provided at the end of the arm 30e. The automatic pipette 30a supplies a culture medium from the reservoir tank 38 via a duct 30g and carries out a pipetting operation. The automatic pipette 30b draws up unneeded culture medium in the culture vessel 6 and the centrifuge receptacle, and discharges it as waste solution into the other reservoir tank 38 via the duct 30g.

The pipetting operation carried out by the automatic pipettes 30a places the chips 34 supplied from the chip supply apparatuses 35 at the tip of the automatic pipette 30a, and suction/ejection is repeated 10 to 20 times to mix the cells and culture medium. By doing so the mixture is uniformly mixed.

After the pipetting operation, the supply robot 30 uses the automatic pipette 30a to draw up the mixture of cells and culture medium separated by the centrifugal separator 31, and supplies it via the opening at the top of the culture vessel 6 mounted on the mounting stage 41.

The used chips 34 are recovered by extracting them in the chip recovery units 51. Therefore, various devices such as the mounting stage 41, the chip supply apparatuses 35, the chip recovery units 51, and an apparatus for supplying cells (not shown in the drawing) from the centrifugal separator 31 are disposed within the operating range of the supply robot 30.

The centrifugal separator 31 receives a centrifuge receptacle containing the cellular suspension mixed by the pipetting action and rotates it at low speed. This allows white blood cells, such as mesenchymal stem cells, contained in bone marrow to be separated from the rest of the liquid and settle. Also, by rotating the cell-containing culture medium supplied from the supply robot 30 at low speed, cells with a high specific gravity suspended in the culture medium are separated from the culture medium and settle.

The dispensing robots 33 each include a horizontally rotatable arm 33a having the automatic pipette 32 to which the chips 34 are removably attached at the tip thereof, and a raising and lowering mechanism 33b for raising and lowering the arm 33a. The dispensing robots 33 supply culture medium or various reagents to the culture vessels 6 transferred by the horizontal transfer mechanism 39. Therefore, various devices such as the mounting stage 41 on the horizontal transfer mechanism 39, the chip supply apparatuses 35, the chip recovery units 51, and the reagent supply apparatuses 36 are disposed within the operating range of the dispensing robots 33.

The chip supply apparatuses 35 contain a plurality of the chips 34, with openings for attaching to the automatic pipettes 30a, 30b, and 32 facing upwards, inside containers 35a which are open at the top. The supply robot 30 and dispensing robots 33 are designed so that chips 34 are attached to the tips of the automatic pipettes 30a, 30b, and 32 simply by inserting the automatic pipettes 30a, 30b, and 32 from the top when new chips 34a are required. The containers 35a are attached to moving mechanisms 35b so that they can be moved mainly in a direction crossing the direction in which the automatic pipettes 30a, 30b, and 32 are moved by the supply robot 30 and the dispensing robots 33. Also, in the chip supply apparatuses 35 that supply the chips 34 to the dispensing robots 33, other moving mechanisms 35c that move the containers 35a in a direction orthogonal to the moving direction of the moving mechanisms 35b are provided. With this configuration, it is possible for the automatic pipettes 30a, 30b, and 32 to access all chips 34 in the containers 35.

The chip recovery units 51 are provided with gripping apparatuses (not shown in the drawings) for gripping the chips 34 at the entrances of the disposal containers 59, and when the chips 34 used in the supply robot 30 and the dispensing robots 33 are inserted into the gripping apparatuses they are gripped. Thus, when the supply robot 30 and the dispensing robots 33 move the automatic pipettes 30a, 30b, and 32 in this state, the used chips 34 are removed from the tips of the automatic pipettes 30a, 30b, and 32, and are collected in the disposal containers 59 via the ducts 60. The disposal containers 59 are placed in the space S222 in a removable manner and can be replaced as needed.

When replacing the ducts 60 and the disposal containers 59, the culture treatment apparatus 50 may be accessed from outside by opening doors (not shown) provided in the side walls 50a and 50b of the culture treatment apparatus 50.

As shown in FIG. 11 for example, the reagent supplying apparatuses 36 each contain a horizontally rotatable table 36a inside a cylindrical casing, and a plurality of cylindrical reagent containers 36b having a fan-shaped bottom surface are mounted on the table 36a around the circumference thereof. The interior of the casing is held at a constant temperature. Various reagents and so forth are held in reagent containers 36b. Examples include MEM (minimum essential medium) forming a necessary culture medium for culturing cells; DMEM (Dulbeccos's Modified Eagle Medium); serums such as FBS (Fetal Bovine Serum) or human serum; enzymes for breaking down proteins, such as trypsin, which release cells in the culture vessel 6; growth factor such as cytokine, which promotes cellular growth during incubation; differentiation-inducing factor such as dexamethasone, which promotes cell differentiation; antibiotics like penicillin-based antibiotics; hormones such as estrogen; and nutrients such as vitamins and so on.

Insertion holes 36 through which the dispensing robots 33 insert chips 34 at the tip of the automatic pipettes 32 are provided in the top surfaces of the casings of the reagent supplying apparatuses 36. These insertion holes 36c are disposed within the operating range of the dispensing robots 33. Also, reagent containers 36b have openings (not shown) at the upper surface thereof, which are disposed at the same position as the insertion holes 36. With this arrangement, by rotating the tables 36 to position the openings of the reagent containers 36b directly below the insertion holes 36 in the casings, the dispensing robots 33 insert the chips 34 at the tips of the automatic pipettes 32 inside the reagent containers 36 from above, which allows the reagents etc. contained inside to be drawn up. Two reagent supplying apparatuses 36 are provided in order to separately handle chemical solutions in the specimen, such as common trypsin, and liquids inherent to the specimen, such as blood serum.

The microscope 37 is used to examine the appearance of cells or the degree of proliferation inside the culture vessel 6 during the culturing process and when replacing the culture medium. In addition, the microscope 37 is used to count the number of cells in the specimen. The microscope 37 is designed so that operations such as adjustment of the XY stage 37 or actuating distance, changing the magnification, and so on can all be carried out remotely. By placing an eyepiece the outside the second space S2, it is possible to examine the condition of cells inside the culture vessel from outside the automatic culture apparatus 21.

The reservoir tanks 38 contain MEM, PBS (phosphor buffered saline), and so on, which can be commonly used in all specimens, and these liquids are supplied to the reagent containers 36b in the reagent supplying apparatuses 36 as required. The reservoir tanks 38 also include a tank serving as a disposal tank for storing disposed culture medium that is discharged when replacing the culture medium.

The horizontal transfer mechanism 39 includes the slider 40, which can be moved horizontally by means of a linear motion mechanism. The mounting stage 41 is mounted on the slider 40, and the culture vessel 6 mounted on the mounting stage 41 can be transferred from the conveyor 29 to the operating region of the dispensing robots 33.

The mounting stage 41 includes a holding mechanism (not shown in the drawings) for holding the culture vessel 6 transferred from the tray 27 on the conveyer 29. An agitator (not shown) for applying vibrations to the culture vessel 6 may also be provided. For example, in addition to a device that oscillates the culture vessel 6 back and forth within a predetermined angular range, a device that applies ultrasonic vibrations or a device that applies vibrations in the horizontal direction may be employed as the agitator.

A control apparatus (not shown) is connected to each of the devices in the automatic culture apparatus 21 according to this embodiment. The control apparatus controls the sequence and timing of the various steps and also keeps a record of the operating history of the devices, and so on.

The operation of the culture treatment apparatus 50 and the automatic culture apparatus 21 according to this embodiment, having the above configuration, will be described below.

To culture bone-marrow mesenchymal stem cells using the automatic culture apparatus 21 according to this embodiment, first, bone marrow taken from a patient is filled into a centrifuge receptacle (not shown) and is placed in the centrifugal separator 31. This step may be performed by the operator or by the supply robot 30. Then, by operating the centrifugal separator 31, bone marrow cells having a high specific density can be collected from the bone marrow.

The collected bone marrow cells are then placed in the culture vessel 6 by the supply robot 30. At this point, by operating the conveyor 29, six empty culture vessels 6 mounted on the tray 27 are delivered from the first space S1 to the second space S2. Among the culture vessels 6 on the tray 27, two culture vessels 6 are lifted from the tray 27 by the robot hand 9 of the handling robot 76, together with the culture-vessel adaptors 1, and are mounted on the mounting stage 41.

According to this embodiment, since the robot hand 9 of the handling robot 76 engages with the protruding part 4 of the culture-vessel adaptors 1 from below, to lift the culture vessels 6, there is no risk of the culture vessels 6 falling, and high-speed handling can thus be realized.

Then, by operating a cover opening/closing device (not shown), covers are removed from the culture vessels 6 on the mounting stage 41 to open them.

When unused chips 34 are disposed within the operating region of the supply robot 30 by operating the moving mechanism 35b of the chip supplying apparatus 35, the supply robot 30 lowers the head 30c by operating the raising and lowering mechanism 30f, receives an unused chip 34 from the chip supplying apparatus 35, which is below the first compartment wall 53, and attaches the chip 34 to the automatic pipette 30a.

In this state, the supply robot 30 is operated to bring the chip 34 at the tip of the automatic pipette 30a into contact with the bone marrow cellular suspension collected in the centrifugal separator 31. Then, by operating the automatic pipette 30a, the bone marrow cells are drawn up into the chip 34. By operating the supply robot 30, the drawn-up bone marrow cells are filled into the culture vessel 6 on the mounting stage 41 via the opening at the top.

Upon completion of filling the bone marrow cells into the culture vessel 6, the supply robot 30 places the chip 34 into the disposal hole 57 formed in the first compartment wall 53 to dispose of it, and it is then recovered by the chip recovery unit 51. The chip 34 disposed through the disposal hole 57 is then placed in the disposal container 59 in the bottommost space S222 via the duct 60.

By operating the raising and lowering mechanism 30f in this state, the head 30c of the supply robot 30 is lowered to receive an unused chip 34 from the chip supplying apparatus 35 below the first compartment wall 53, and the chip 34 is attached to the automatic pipette 30b. In this state, the supply robot 30 is operated to supply DMEM or PBS (phosphor buffered saline) held in the reservoir tank 38 to the culture vessel 6, via the chip 34 at the tip of the automatic pipette 30b.

Next, by operating the horizontal transfer mechanism 39, the culture vessels 6 containing bone marrow cells are transferred horizontally together with the mounting stage 41, and is positioned within the operating region of the dispensing robots 33. By operating the automatic pipette 32 having an unused chip 34 received from the chip supplying apparatus 35 attached to the tip thereof, the dispensing robot 33 draws up a suitable amount of DMEM, blood serum, or various reagents from the reagent container 36b in the reagent supplying apparatus 36, and thereafter transfers the culture vessel 6 upwards to dispense the drawn-up liquid into the culture vessel 6. When drawing up the blood serum and reagents, the chip 34 is replaced with an unused chip 34 from the chip supplying apparatus 35 each time the reagents etc. are drawn up. By doing so, bone marrow cells in a suitable culture medium exist in a mixed state in the culture vessel 6. To uniformly distribute the bone marrow cells in the culture medium the mounting stage 41 may be operated to vibrate together with the culture vessel 6.

Subsequently, when all processing has been completed, the horizontal transfer mechanism 39 is operated to move the culture vessel 6 adjacent to the conveyor 29, whereat the cover opening/closing mechanism and the transfer mechanism are operated again to seal the opening with the cover 3b, and in this state, the culture vessel 6 is returned to the tray 27 using the handling robot 76.

When predetermined processing has been completed for all culture vessels 6 on the tray 27, the conveyer 29 is operated to move the culture vessels 6 mounted on the tray 27 from the second space S2 into the central space S12 of the first space S1.

By operating the transfer robot 25 in this state, the tray 27 is lifted by the hand 25. Then, once it has been transferred to a point in front of the incubation chamber 24 for storing that tray 27, the door 24a of the incubation chamber 24 is opened, and the tray 27 is placed onto empty tray holding members 24 using the transfer robot 25. Then, once the door 24a is closed, the incubation conditions inside the incubation chamber are again kept constant, which allows the cells to be cultured.

It goes without saying that the order in which the bone marrow cells, DMEM, blood serum, and various reagents are filled or drawn up may be changed as desired.

In the same way as described above, when replacing the culture medium and the container, by operating the transfer robot 25, which is disposed outside the incubation chamber, the culture vessel 6 inside the incubation chamber 24 is taken out together with the tray 27 and is delivered from the first space S1 to the second space S2. In the second space S2, trypsin is dispensed into the culture vessels 6, and once cells inside the culture vessel 6 are released, they are placed in the centrifugal separator 31 by operating the supply robot 30, and only the required part, such as mesenchymal stem cells, is collected. The other processing steps are similar to that described above.

Then, by carrying out a culturing process for a predetermined period through a plurality of culture medium replacements and container replacements, the mesenchymal stem cells multiply to produce a sufficient number of cells. To determine whether or not a sufficient number of cells have been produced, the culture vessel 6 having the mesenchymal stem cells adhering to the bottom surface is transferred to the microscope 37 by operating the supply robot 30, and examination or measurement is carried out to determine the degree of proliferation of the cells. The culture vessels 6 mounted on the tray 27 may contain the same types of specimen, or culture vessels 6 containing different specimens may be mixed. Also, the culture vessels 6 mounted on the mounting stage 41 may contain the same type of specimen, or culture vessels 6 containing different specimens may be mixed.

Whether to proceed to the next stage based on the determined degree of proliferation of the cells or whether to carry out one more proliferation cycle may be determined automatically.

By doing so, with the automatic culture apparatus 21 according to this embodiment, it is possible to automatically culture a desired number of mesenchymal stem cells from bone marrow taken from a patient. After obtaining a sufficient number of mesenchymal stem cells, a tissue filling material such as calcium phosphate and a differentiation-inducing factor such as dexamethasone are introduced into the culture vessels, and by repeating the culture process, a tissue filling material that can compensate for tissue deficiencies may be produced.

In this case, with the automatic culture apparatus 21 according to this embodiment, no mechanical parts for removing the culture vessels 6 exist inside the incubation chambers 24. In other words, only the tray holding members 24c for supporting the trays 27 are provided inside the incubation chambers 24 and the mechanical parts for removing the culture vessels 6 are completely integrated in the transfer robot 25, which is disposed outside the incubation chambers 24. Thus, after completing extraction and insertion of the trays 27, the transfer robot 25 can remain entirely outside the incubation chambers 24.

Therefore, when the doors 24a are closed, no mechanical parts exist inside the incubation chambers, and therefore, no dust is produced inside the chambers, such as the kind of dust normally produced when operating mechanical parts. In addition, although a temperature of 37±0.5° C., a humidity of 100%, a CO₂ concentration of 5%, and so forth are maintained inside the incubation chambers, since there are no mechanical parts, there are no problems such as corrosion, despite these conditions. Furthermore, even if the doors 24a are opened, since only the end of the hand 25c of the transfer robot 25 enters the incubation chambers 24, no rotating mechanism or sliding mechanism actually enters the incubation chambers 24. As a result, dust contamination of the incubation chambers 24 can be controlled, which allows a high degree of cleanliness inside the incubation chambers 24.

The incubation chambers 24 may any type of incubator used for culturing, such as CO₂ incubators, multi-gas incubators, standard incubators, or refrigerated incubators. Alternatively, they may be a combination of any of the above.

With the culture treatment apparatus 50 and the automatic culture apparatus 21 according to this embodiment, the second space S2 of the culture treatment apparatus 50 is divided into the upper space S21 and the lower space S22 by the first compartment wall 53. Also, the air cleaning unit 52 for producing a downward flow of clean air is provided in the upper space S21. The ventilation holes 58 are provided in the first compartment wall 53 close to the side walls 50a and 50b thereof. In addition to the ventilation holes 58, the through-holes 56, 57, etc. passing through various devices are formed in the first compartment wall 53. However, by ensuring that the ventilation cross-section of the ventilation holes 58 is sufficiently larger than the ventilation cross-section of the through holes 56, 57, etc., the airflow can be made to pass through the ventilation holes 58.

Therefore, the clean airflow coming down through the upper space S21 goes towards the side walls 50a and 50b close to the first compartment wall 53, and flows to the lower space S22 via the ventilation holes 58. As a result, an airflow that pushes dust floating in the upper space S21 downwards is made to smoothly flow to the lower space S22 without being retained in the corners near the side walls 50a and 50b of the upper space S21.

Furthermore, with the culture treatment apparatus 50 and the automatic culture apparatus 21 according to this embodiment, in the upper space S21 in which the culture vessels 6 are transferred with their covers removed, only the parts that are necessary for transferring the culture vessels 6, such as the mounting stage 41 and the XY table 37a of the microscope 37, parts that need to access the culture vessels 6 from the opening at the top, such as the supply robot 30 and the automatic pipette 32 of the dispensing robot 33, and the light source unit of the microscope are disposed, and other parts are disposed in the lower space S22. Therefore, the generation of dust in the upper space S21 can be kept to a minimum, and the risk of dust contamination of the culture vessels 6 is thus reduced.

In particular, since devices having a high likelihood of producing dust, for example, the centrifugal separator 31, the disposal container 59, the exhaust fan 61, and so on, are disposed in the lower space S22, and furthermore, in the lowermost space S222 partitioned by the second compartment wall 54, any dust produced does not contaminate the upper space S21. Also, the air in the space S222 is drawn in by the exhaust fan 61 and is discharged outside the culture treatment apparatus 50 after the dust is removed by the HEPA filter 62. Therefore, the degree of cleanliness in the upper space S21 is maintained at an extremely high level.

Since ventilation holes 63 are also provided in the second compartment wall 54 parallel to the side walls 50a and 50b, an airflow including dust from the upper space S21 can be made to smoothly flow to the space S222 without spreading out in the space S221.

Also, by replacing the removable disposal containers 59, which contain used chips 34 to which culture medium and cells are attached, either as needed or at specified intervals, it is possible to return to a high degree of cleanliness in the lower space S22. By also removing the ducts 60 through which the used chips 34 pass when they are disposed of into the disposal containers 59 in order to replace them or clean them out, either as needed or at specified intervals, it is possible to improve the level of cleanliness.

Since the automatic culture apparatus according to this embodiment is provided with the air cleaning unit 26 at the top of the central space S12 where the transfer robot 25 is disposed, a degree of cleanliness can always be maintained in the central space S12 too. Therefore, even when the doors 24a of the incubation chambers 24 are opened, it is possible to keep contamination of the incubation chambers 24 by dust to a minimum.

Therefore, with the automatic culture apparatus 21 according to this embodiment, the risk of contamination of the cells in the culture medium due to dust or the like is reduced, which affords an advantage in that stable and effective cell culturing can be realized.

The present invention is not limited to the structures described in the above embodiments. That is to say, the configuration and number of the incubation chambers 24, the transfer robot 25, the supply robot 30, the dispensing robots 33, and various other devices is not limited, but may be set as desired according to the application.

Instead of cytokine, any material that encourages growth may be used as the growth factor, including, for example, platelet concentrate, BMP, EGF, FGF, TGF-β, IGF, PDGF, VEGF, HGF or a combination thereof. Also, instead of a penicillin-based antibiotic, any antibiotic may be employed, including cephems, macrolides, tetracyclines, fosfomycins, aminoglycosides, new quinolones, and so on.

The automatic culture apparatus according to the present invention is not limited to culturing of bone-marrow mesenchymal stem cells. Cells taken from various tissues or established cell lines may be cultured.

As the tissue filling material, instead of calcium phosphate, any material having compatibility with tissue may be used, preferably bioabsorbable material. In particular, porous ceramic having biocompatability, collagen, polylactic acid, polyglycolic acid, hyaluronic acid, or any combination of these may be used. In addition, a metal like titanium may be used. Furthermore, the tissue filling material may be in granular form or in bulk form.

1. A culture-vessel adaptor that is detachably fixed to the exterior of a culture vessel capable of holding a culture medium containing a specimen, the culture-vessel adaptor comprising:
a vessel mounting part where the culture vessel is mounted; and
a protruding part disposed so as to extend outward from a side surface of the culture vessel when the culture vessel in mounted in the vessel mounting part.

2. The culture-vessel adaptor according to claim 1, further comprising:
a peripheral wall that is connected to the vessel mounting part around the entire circumference of the vessel mounting part and that is disposed at a certain distance outside the side surface of the culture vessel when the culture vessel is mounted in the vessel mounting part.

3. The culture-vessel adaptor according to claim 1, wherein the protruding part is formed in the shape of a flange.

4. The culture-vessel adaptor according to claim 1, wherein a fitting portion for fitting the culture vessel is provided in the vessel mounting part.

5. The culture-vessel adaptor according to claim 1, wherein the culture-vessel adaptor is formed of a heat-resistant plastic material.

6. The culture-vessel adaptor according to claim 1, wherein at least the vessel mounting part is formed of a transparent material.

7. The culture-vessel adaptor according to claim 1, wherein a through-hole is formed in the vessel mounting part.

8. A culture treatment apparatus comprising:
a treatment chamber containing a treatment apparatus that opens a lid of a culture vessel containing a specimen and that performs predetermined treatment on the specimen in the culture vessel; and
a handling apparatus, provided in the treatment chamber, for transferring a culture-vessel adaptor according to claim 1, in which the culture vessel is mounted, by engaging with the protruding part from below.
